(19) United States
(12) Patent Application Publication
(10) Pub. No.: US 2021/0162197 A1
(43) Pub. Date: Jun. 3, 2021

(54) PERCUTANEOUS CONDUIT DEPLOYMENT METHOD AND INSTRUMENTS THEREFOR

(71) Applicant: CARDIAC ASSIST HOLDINGS, Ann Arbor, MI (US)

(72) Inventors: Allen B. Kantrowitz, Ann Arbor, MI (US); Barry N. Gellman, Ann Arbor, MI (US); Kurt A. Dasse, Ann Arbor, MI (US)

(73) Assignee: CARDIAC ASSIST HOLDINGS, Ann Arbor, MI (US)

(21) Appl. No.: 17/045,207

(22) PCT Filed: Apr. 4, 2019
(86) PCT No.: PCT/US2019/025874

§ 371 Date: Oct. 5, 2020

Related U.S. Application Data
(60) Provisional application No. 62/652,368, filed on Apr. 4, 2018.

(51) Int. Cl. A61M 60/165; A61M 60/88; A61L 33/00; A61L 31/10; A61B 90/00

(52) U.S. Cl. CPC A61M 60/165; A61M 60/88; A61L 33/0011; A61B 2090/3937; A61B 90/39; A61M 2205/0238; A61L 2420/08; A61L 31/10

(57) ABSTRACT

A deployment system and method of use thereof is provided for an interior driveline that mitigates the risk of infections for a variety of implanted medical appliances with a percutaneous conduit. The interior driveline and driveline deployment system allow for the deployment of a driveline and an optional corresponding percutaneous access device (PAD) from within the tissue layers below the dermis, prior to exiting the body of a patient. The interior introduction of the driveline and corresponding PAD precludes entrainment introduction of exogeneous pathogens associated with the traditional approach for insertion of a driveline with an exterior to interior directionality relative to the subject corpus.

### RELATED APPLICATIONS

This application claims priority benefit of U.S. Provisional Application Ser. No. 62/652,368 filed 4 Apr. 2018, the contents of which are hereby incorporated by reference.

### FIELD OF THE INVENTION

The present invention in general relates to medical devices and systems and in particular, to create an appropriate pathway through the skin to serve as a conduit through which a medical appliance or a percutaneous access device (PAD) can be implanted and a method of deployment thereof.

### BACKGROUND OF THE INVENTION

Heart disease is one of the leading causes of death. Currently, medical science cannot reverse the damage done to the cardiac muscle by heart disease. One solution for such patients is a heart transplant. However, the number of cardiac patients in need of a heart transplant far exceeds the limited supply of donor hearts available.

The scarcity of human hearts available for transplant, as well as the logistics necessary to undertake heart transplant surgery, makes an implantable cardiac assist device a viable option for many heart patients. A blood pump can be surgically implanted, or adjacent to, the cardiovascular system to augment the pumping action of the heart. The blood pump is sometimes referred to as a mechanical auxiliary ventricle assist device, dynamic aortic patch, balloon pump, mechanical circulatory assist device, or a total mechanical heart. Alternatively, the blood pump can be inserted endovascularly.

Typically, the blood pump systems include a driveline that serves as a power and/or signal conduit between the blood pump internal to the patient and a controller/console external to the patient. Often, a percutaneous access device (PAD) can be surgically implanted at the location in the skin where the driveline penetrates the skin to provide a through-the-skin coupling for connecting the supply tube to an extra-corporeal fluid pressure source. Electrical leads from electrodes that may be implanted in the myocardium are likewise brought out through the skin by means of the PAD. The aortic valve status or any cardiovascular parameter that is associated with this status can be employed to control the fluid pressure source to inflate and deflate the inflatable chamber in a predetermined synchronous relationship with the heart action.

The surface of the driveline, or of the optional PAD used in cardiac assist systems may have characteristics which promote the formation of a natural biologic seal between the skin and the device to form a barrier to microbial invasion into the body at the skin penetration site. More generally, medical appliances which are implanted so as to cross the skin surface and therefore violate the “barrier function” of the skin, may also illustratively be used for other medical purposes including peritoneal dialysis catheters, chronic indwelling venous access catheters, neurologic prostheses, osseointegrated prostheses, drug pumps, and other treatments that require skin penetration.

FIG. 1 illustrates wearable and implanted components of an exemplary prior art cardiac assist system. A PAD 10 serves as an attachment point for an external supply line 12 that supplies air or fluid from a wearable external drive unit (EDU) 14. The EDU 14 is powered by a wearable battery pack 16. Inside the body of the patient, a drive line 18 is attached to the PAD 10 and provides an air or fluid conduit to a cardiac assist device 20.

While conventional aortic balloon pumps and other implanted assist devices are well known to the art, driveline infection remains one of the most frequent and costly adverse events, associated with implanted assist devices, at the percutaneous access device (PAD). Driveline infections may be predisposed to systemic infections due to ascending microbial invasion.

Ventricular Assist Device (LVAD) driveline infections (DLI) are the most common type of infection associated with implantable pumps. These infections occur at the skin penetration site because current devices require an external power source with energy supplied via a tunneled percutaneous driveline. Driveline infections frequently occur because the driveline exit site creates a conduit for entry of bacteria. DLI, along with gastrointestinal bleeding (GIB) and stroke, are the leading causes of unplanned readmission for patients with an LVAD

There is a continuing need for internal drivelines for implanted devices and methods of implantation thereof that significantly minimize or inhibit the risk of driveline and exit site infections

### SUMMARY OF THE INVENTION

A system for deploying a percutaneous conduit from within the skin of a patient in the subcutaneous layer includes a tunneler having an elongated passer section, the elongated passer section having a proximal end terminated with an attachment feature for the percutaneous conduit and a distal end terminated in a tunneling head. A central needle is in the tunneling head in a coaxial channel in the elongated passer section containing a wire for advancement of the central needle. A cylindrical trephine blade is attached to the central needle, where when the central needle is advanced through and pierces an outer layer of the patient's skin in an outward direction away from the patient, and the central needle defines a longitudinal axis of trephination or cylindrical cut of the cylindrical trephine blade in the patient's skin that is used for placement of a medical appliance or a percutaneous access device (PAD).

A system for deploying a percutaneous conduit from within the skin of a patient in the subcutaneous layer includes a tunneler having an elongated passer section, the elongated passer section having a proximal end terminated with an attachment feature for the percutaneous conduit and a distal end terminated in a tunneling head. A central needle with a cam is in a central channel of the tunneling head, where a lower chamber in the form of a semicircle off of the central channel allows for partial upward movement of the cam to a first stop, and an upper chamber in the form of a semicircle off of the central channel that allows for partial upward movement of the cam to a second stop. A coaxial channel in the elongated passer section contains a wire for advancement and twisting of the central needle. A cylindrical trephine blade is actuated by the cam of the central needle in the upper chamber, where when the central needle is advanced through and pierces an outer layer of the patient's skin in an outward direction away from the patient, and the central needle defines a longitudinal axis of trephination or cylindrical cut of the cylindrical trephine blade in the patient's skin that is used for placement of a medical appliance or a percutaneous access device (PAD).

A method for deploying a percutaneous conduit from within the skin of a patient in the subcutaneous layer includes making a sub cut in the subcutaneous layer, inserting a tunneler in the sub cut, and advancing the tunneling head to form a tunnel in the subcutaneous layer to an exit location. Subsequently, a central needle is deployed, or following a pre-positioned central guidewire, to pierce through the skin of the patient. A cylindrical trephine blade is deployed to make a cylindrical cut in the skin, and the tunneler is pulled or pushed out of the patient to expose the driveline and for positioning the medical appliance or PAD in the cylindrical cut. The tunneler is then detached from the driveline

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which like reference numerals refer to like parts throughout the several views, and wherein:

FIG. 1 illustrates prior art wearable and implanted components of a cardiac assist system with a percutaneous access device (PAD) and internal driveline;

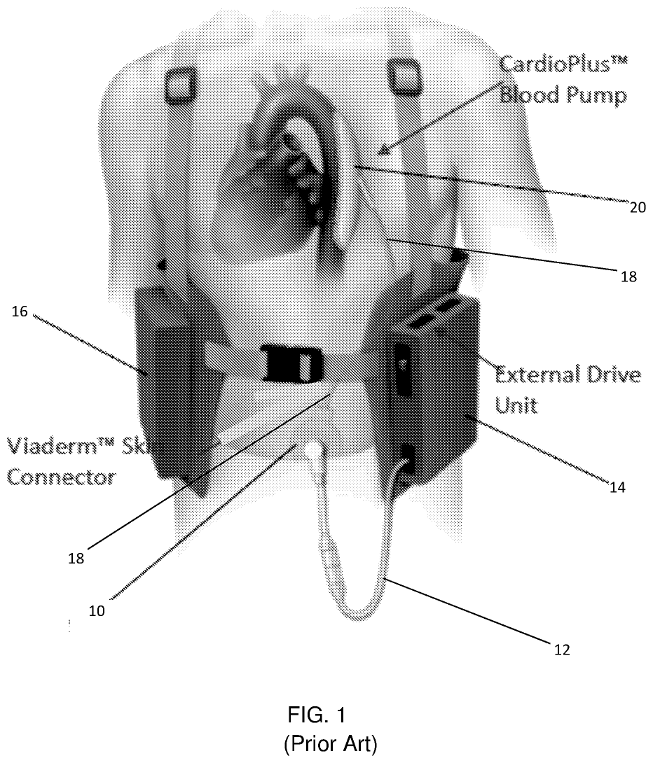

FIGS. 2A-2E are a series of partial cutaway side perspective views showing the internal implantation and deployment of an internal driveline and an optional PAD in the subcutaneous layer below the dermis and above the fascia layer in accordance with embodiments of the invention;

FIG. 3 is an exploded sectioned view of a tunneler tip and corresponding central lead needle, and cylindrical trephine blade in accordance with an embodiment of the invention;

FIGS. 4A-4D are a series of side perspective cross-sectional views showing the internal implantation and deployment of an internal driveline using the tunneler tip of FIG. 3 in accordance with an embodiment of the invention;

FIGS. 5A-5E are a series of detailed cross-sectional side views of the tunneler tip of FIG. 3 illustrating the deployment of the central lead needle and cylindrical trephine blade in a patient according to an embodiment of the invention;

FIG. 6 is a side perspective cross-sectional view of a quick locking fastener according to an embodiment of the invention; and

FIG. 7 is a side perspective cross-sectional view of a tunneler to central lead needle locking mechanism according to an embodiment of the invention.

### DETAILED DESCRIPTION OF THE INVENTION

An interior driveline and driveline deployment system according to the present invention has utility to mitigate the risk of infections for a variety of implanted medical appliances with a percutaneous conduit. While the present invention is further detailed with respect to a cardiac assist device driveline, it is appreciated that the present invention informs the deployment of various implanted medical devices having a percutaneous conduit, such devices also illustratively include a percutaneous central catheter (PICC), a colostomy port, a peritoneal dialysis catheters, a dialysis conduit, an insulin pump, neurologic prostheses, osseointegrated prostheses, drug pumps, and other treatments that require skin penetration or any connection between an internal organ or cavity and an extracorporeal device. Embodiments of the inventive interior driveline and driveline deployment system allow for the deployment of a driveline and an optional corresponding percutaneous access device (PAD) from within the tissue layers below the dermis, prior to exiting the body of a patient. It has been surprisingly found that deployment related infections can be effectively eliminated with resort to the present invention. The counterintuitive interior introduction of the driveline and corresponding PAD of the present invention thereby precludes entrainment introduction of exogeneous pathogens associated with the traditional approach for insertion of a driveline with an exterior to interior directionality relative to the subject corpus.

In a specific embodiment the interior driveline (DL) or percutaneous conduit incorporates a polymeric or polyester velour. In specific inventive embodiments the textured contacting outer surfaces is based on Integrally Textured Polymer (ITP) formed as a membrane and in an exemplary embodiment an Integrally Textured (IT) Polyurethane. Other suitable materials for an ITP illustratively include polyamides, polyimides, polyesters, polycarbonates, copolycarbonate esters, polyethers, polyetherketones, polyetherimides, polyethersulfones, polysulfones, polyvinylidene fluoride, polybenzimidazoles, polybenzoxazoles, polyacrylonitrile, cellulosic derivatives, polyazoaromaties, poly(2,6-dimethylphenylene oxide), polyphenylene oxides, polyureas, polyurethanes, polyhydrazides, polyazomethines, polyacetals, cellulose acetates, cellulose nitrate, ethyl cellulose, styrene-acrylonitrile copolymers, brominated poly(xylylene oxide), sulfonated poly(xylylene oxide), tetrahalogen-substituted polycarbonates, tetrahalogen-substituted polyesters, tetrahalogen-substituted polycarbonate esters, polyquinoxaline, polyamideimides, polyamide esters, polysiloxanes, polyacetylenes, polyphosphazenes, polyethylenes, polyphenylenes, poly(4-methylpentene), poly(trimethylsilylpropyne), poly(trialkylsilylacetylenes), polyureas, polyurethanes, blends thereof, block copolymers thereof; a fiber or particle filled forms of any of the aforementioned.

Furthermore, the surface of the medical appliance or PAD used in inventive embodiments promotes the formation of a natural biologic seal between the skin and the device to form a barrier to microbial invasion into the body via the skin penetration site. Embodiments of the PAD may also illustratively be used cardiac assist devices and for other devices including peritoneal dialysis catheters and chronic indwelling venous access catheters that require skin penetration. The percutaneous access device used with some inventive embodiments of the percutaneous conduit are pre-coated with a recipient's dermal fibroblasts. These dermal fibroblasts inhibit epidermal down growth, preventing sinus tract formation along the driveline; and an environment that supports microbial growth.

Embodiments of the percutaneous conduit may be treated with a primary coating to promote long-term stability and therefore any implanted device anchored thereto. Such coating substances illustratively include heparin, antibiotics, radiopaque agents, anti-thrombogenic agents, anti-proliferative agents, pro-proliferative agents, anti-angiogenic agents, and pro-angiogenic agents; each alone, or in combination. It is further appreciated that a secondary coating overlying the first coating is provided to promote sustained release of the underlying coating substance. Such secondary coatings illustratively include polylactic acid, polyglycolic acid, polyethylene oxide, polycaprolactone, polydioxanones, combinations thereof, and co-polymers thereof.

In certain inventive embodiments, the interior driveline may be formed from a material that induces immunocompatible granulation tissue overgrowth thereon. Coatings operative herein illustratively include poly-L-lysine (PLL), polylmethyl coguanidine-cellulose sulphate (PMCG)-CS/PLL-sodium alginate (SA), polyethylenimine, poly(dimethyldiallylammonium chloride), chitosan, polyacrylacid, carboxymethylcellulose, cellulose sulfate, pectin, and combinations thereof to form multilayers. It is appreciated that such coatings are readily impregnated with compounds that reduce the immune cascade or alternatively, enhance the inflammatory cascade, these illustratively include heparin and factor H.

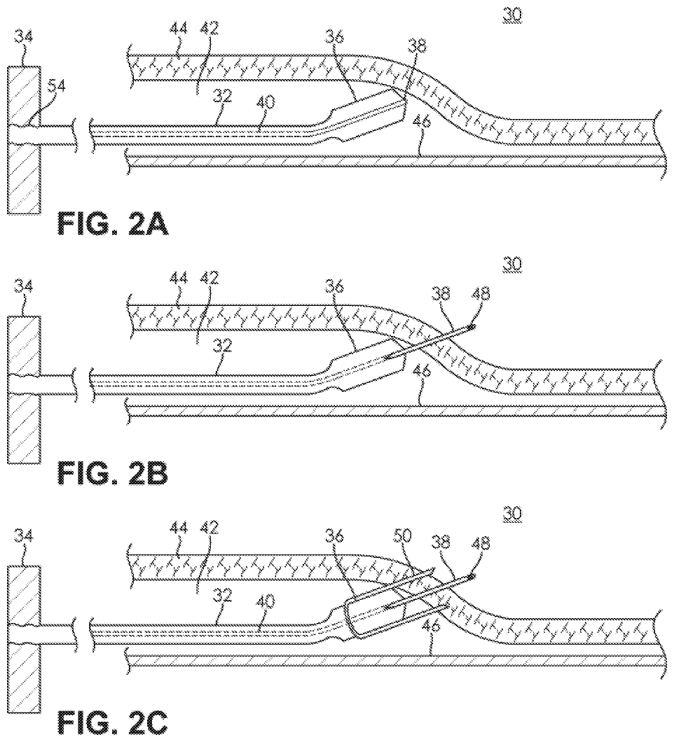

Referring now to the figures, FIGS. 2A-2E are a series of partial cutaway side perspective views showing a system 30 for the internal implantation and deployment of an embodiment of the internal driveline 52 and an optional PAD 62 in the subcutaneous layer 42 below the dermis 44 and above the fascia layer 46. In FIG. 2A a sub cut is made for a subcutaneous tunnel that is formed in the subcutaneous layer 42 when a tunneler is inserted in and advanced through the sub cut. The tunneler has an elongated passer section 32 with a proximal end terminated with an attachment feature 54 illustratively including screw threads, and a distal end terminated in a tunneling head 36. The attachment feature 54 may be used to secure a primary “T” handle 34 that is used to push the tunneler through the subcutaneous layer 42 to a desired exit site. The elongated passer section 32 may have a coaxial channel with a wire 40 therein. The wire 40 may be used to advance a central needle 38 connected to the distal end of the wire 40 in the tunneling head 36. In FIG. 2B the central needle 38 is advanced through and pierces the dermis 44 in an outward direction away from the patient. The central needle 38 defines a longitudinal axis of the trephination or cylindrical cut in the dermis 44 where the medical appliance, with the optional PAD 62, will be inserted as shown in FIG. 2E. In FIG. 2C as the central needle 38 is further advanced through the dermis 44, and the cylindrical trephine blade 50 begins to deploy from the tunneling head 36 and pierce the dermis 44. In FIG. 2D a secondary “T” handle 56 is attached via an engagement feature or eyelet 48 in the central needle 38 and is used to pull and twist (arrow 60) the trephine blade 50 through the dermis 44 with the removed cored tissue 58 held by the tunneling head 36 within the cylindrical area of the trephine blade 50. Also shown in FIG. 2D, a driveline 52 is attached to the attachment feature 54 of the elongated passer 32 and pulled through the subcutaneous layer 42 to the newly created skin opening. As shown in FIG. 2E, the driveline 52 is continued to be pulled out of the patient until the prior secured optional PAD 62, which concentrically surrounds the driveline 52, is pulled into the newly created skin opening. At this point the driveline 52 is in the final desired position. The driveline 52 is subsequently attached to the implanted device (not shown). It is appreciated that the eyelet 48 is amenable to engage a cam (not shown) or similar engagement mechanism to secure the secondary T handle 56. Additionally, not shown, a hollow channel or eyelet can allow a guidewire to serve to define a percutaneous path to be followed thereafter by the needle 38.

In some inventive embodiments, a tracking system is deployed that includes two or more receivers to detect the position of fiducial markers (e.g., retroreflective spheres, active light emitting diodes (LEDs), or radiofrequency identification tags) arranged on a subject body and the inventive tunneler apparatus. The fiducial markers collectively define a fiducial marker array. In some inventive embodiments, each fiducial marker has a unique arrangement of fiducial markers, or a unique transmitting wavelength/frequency to distinguish one marker array from another. An example of an optical tracking system operative herein is described in U.S. Pat. No. 6,061,644. With resort to a tracking system computer that includes tracking hardware, software, data, and utilities to determine the position and orientation (POSE) of objects (e.g., receipt tissue and instruments) in a local or global coordinate frame, an inventive method is amenable to being conducted under autonomous or semiautonomous robotic control. The POSE of the objects is collectively referred to herein as POSE data, where this POSE data may be communicated to the device computer through a wired or wireless connection. Alternatively, the device computer may determine the POSE data using the position of the fiducial markers detected from the optical receivers directly.

The POSE data is determined using the position data detected from the receivers and operations/processes such as image processing, image filtering, triangulation algorithms, geometric relationship processing, registration algorithms, calibration algorithms, and coordinate transformation processing. For example, the POSE of an optically tracked digitizer probe with an attached probe fiducial marker array is calibrated such that the probe tip is continuously known as described in U.S. Pat. No. 7,043,961. Registration algorithms may be executed to determine the POSE and coordinate transforms between recipient tissue, pre-operative tissue data, a fiducial marker array, a surgical plan, a surgical robot, and/or tracking system using the registration methods as described above.

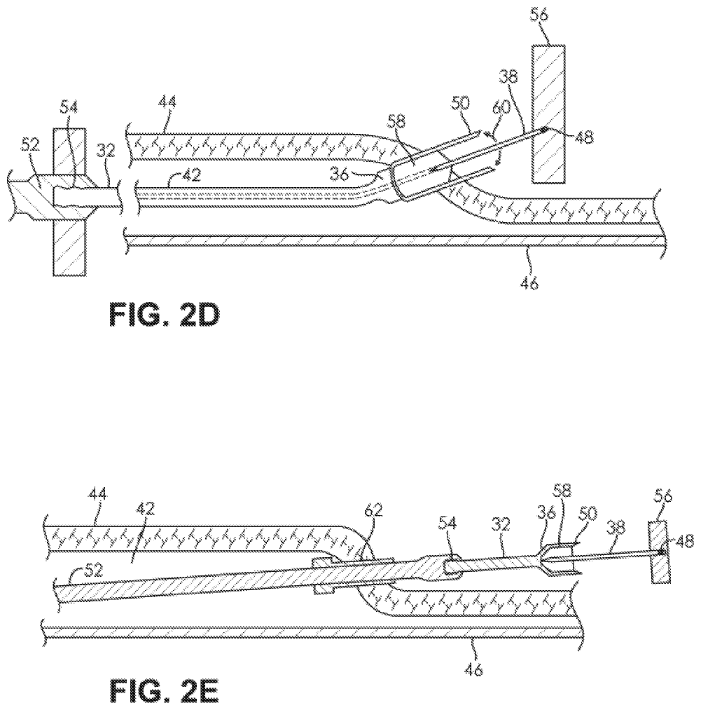

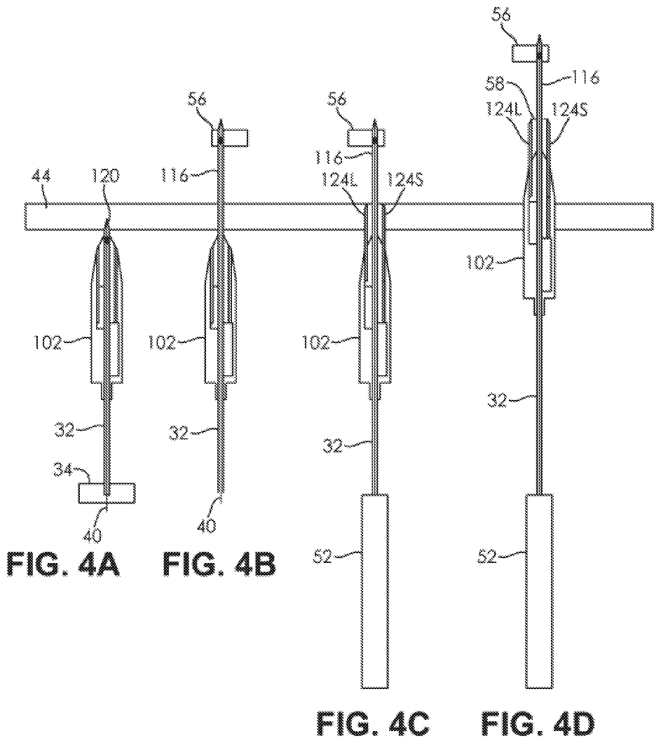

FIG. 3 is an exploded sectioned view of an inventive embodiment of a tunneler head 100 and corresponding tunneler tip 102, central lead needle 116, and cylindrical trephine blade 124 (shown in longitudinal section with a long side (124L) and short side (124S). The cylindrical trephine blade 124 is designed to be carried within tunneler tip 102 of the tunneler head 100 in slots (112L, 122S). The cylindrical trephine blade 124 is secondarily deployed to incise a core of dermis/epidermis at an exit site. The anti-incision end (flat end) of the long side (124L) engages with a cam 122 on the central needle 116 as is best shown in FIGS. 5D and 5E. The tunneler tip 102 has a central channel 104 that houses the central needle 116. A lower chamber 106 in the form of a semicircle off of the central channel 104 allows for partial upward movement of the cam 122 with a first stop 108. In order to further advance the central needle 116, the cam 122 needs to be rotated into the semicircle shaped upper chamber 110. As best shown in FIGS. 5D and 5E, the cam 122 then engages the long side (124L) of the cylindrical trephine blade 124 to provide secondary deployment of the cylindrical trephine blade 124 from slots (112L, 112S) until a second stop 114 is reached that halts further advancement of the central needle 116 and the cylindrical trephine blade 124.

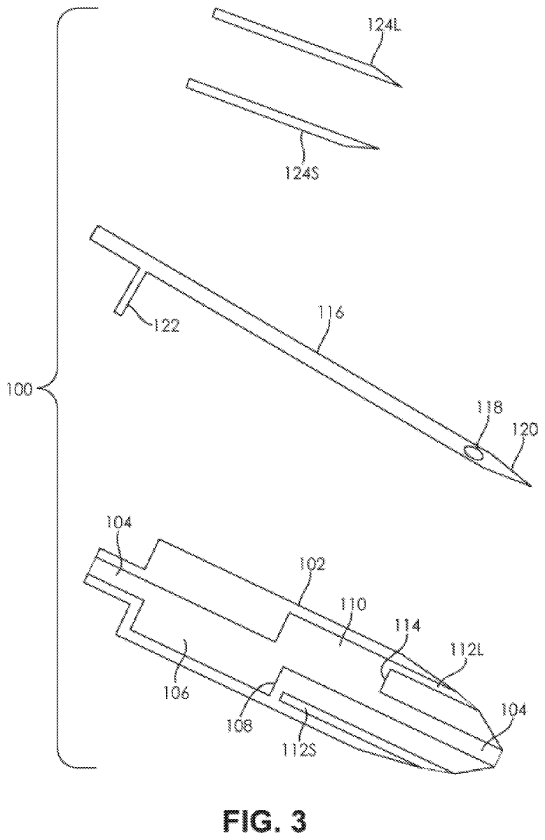

FIGS. 4A-4D are a series of side perspective cross-sectional views showing the internal implantation and deployment of an internal driveline 52 using the tunneler tip 102 of FIG. 3. In FIG. 4A the primary “T” handle 34 is connected to the distal end of the elongated passer 32, while the proximal end of the passer 32 is connected to the tunneler tip 102 with the tip 120 of the central needle 116 beginning to pierce the dermis 44 of the patient. In FIG. 4B wire 40 is advanced to push the central needle 116 through the dermis 44. The primary “T” handle 34 is removed from the distal end of the passer 32, and a secondary “T” handle 56 is attached to the central needle 116 via the engagement feature or eyelet 118 to pull the central needle 116 completely through the dermis 44. FIG. 4C shows the deployment of the cylindrical trephine blade 124 into the dermis 44, and the attachment of the internal driveline 52 to the distal end of the passer 32. FIG. 4D illustrates the passing of the tunneler tip 102 completely through the dermis 44 with a cored section of removed skin 58 held by the cylindrical trephine blade 124.

FIGS. 5A-5E are a series of detailed cross-sectional side views of the tunneler tip 102 of FIG. 3 illustrating the deployment of the central lead needle 116 and cylindrical trephine blade 124 in a patient. FIG. 5A illustrates the tip 120 of the central needle 116 just prior to piercing the dermis 44. In FIG. 5B, the cam 122 advances in the lower chamber 106 until the first stop 108 with the central needle 116 fully passing through the dermis 44. In FIG. 5C, the secondary “T” handle 56 is joined to the central needle 116 via an engagement feature or eyelet 118. In FIG. 5D, the central needle 116 is twisted (arrow 126) with the secondary “T” handle 56 to rotate the cam 122 into the upper chamber 110 to engage the long side of the cylindrical trephine blade 124L. In FIG. 5E the central needle 116 is pulled with the secondary “T” handle 56 to expose the cylindrical trephine blade 124 and to create a trephination or cylindrical cut in the dermis 44 where the PAD will be placed.

It is appreciated that while the cylindrical trephine is described as being advanced through the dermis with a twisting or rotating action, a unidirectional or oscillating action, may be introduced via mechanisms contained in the tunneler itself, or a detachable external or internal rotational mechanism. In specific inventive embodiments the size of the cylindrical trephine is appropriately sized to create a slight interference between the dermal/epidermal trephination incision and the surface of the medical appliance or PAD to encourage and generate tissue ingrowth into the device. Furthermore, not to be limited to a specific theory, but it is believed that potential gaps between the trephination incision and the surface of the medical appliance or PAD encourage the formation of reservoirs which serve to accumulate reactive wound fluids containing dissolved molecules utilized by bacteria to support infective colonization and thereby interfere with the rapid integration of dermal fibroblasts with the surface of the medical appliance or PAD.

In a specific inventive embodiment, in lieu of the central flat or round needle, a guide wire can be separately placed to pierce the skin at the planned skin exit site and a hollow channel within the tunneler head may then follow the pathway of the guidewire outwardly from the patient, where the pathway may be at any arbitrary angle of incidence to the epidermis. In other words, the needle or guidewire may be placed from external-to internal or from internal-to-external, but the remainder of the tunneler, trephine, medical appliance or PAD are passed from the internal-to-external.

In a specific inventive embodiment, a collapsible/expandable trephine with a cannulated central channel can, in its collapsed state, follow the guidewire from outside the skin, past the epidermis, at any arbitrary angle of incidence to the epidermis, and dermis into the subcutaneous tissue, thence open to an expanded state, and subsequently can be pulled externally through the subcutaneous tissue layer/dermis layer and epidermis layer. Thusly, a skin trephination can be excised in a fashion which will provide accurate intimate coaptation of skin layers against the medical appliance in the service of promoting healing between skin layers and the medical appliance.

While it is appreciated that a guidewire introduced from external-to-internal might contaminate the needle tract from the introduced external wire, it is understood that this contaminated skin would be excised by the cylindrical trephine with the core of removed skin tissue. Moreover, while the needle or guidewire may be placed from external-to internal or from internal-to-external, the remainder of the tunneler, trephine, medical appliance or PAD are passed, with appropriate sheathing or other protective mechanisms, either from the internal-to-external direction or the external-to-internal direction.

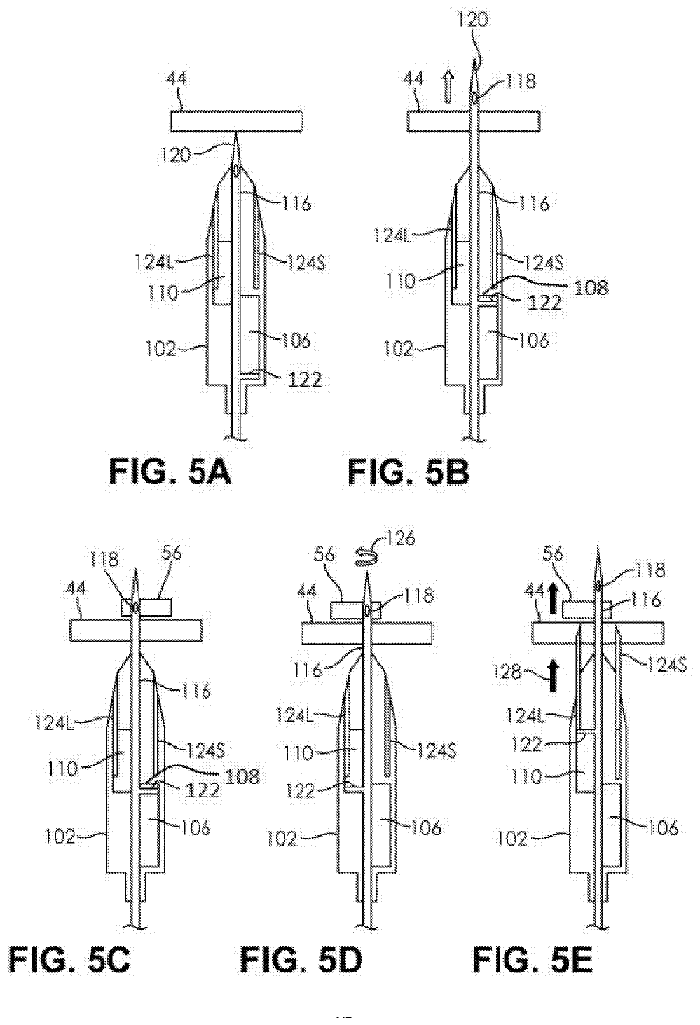

FIG. 6 is a side perspective cross-sectional view of a quick locking fastener 130.

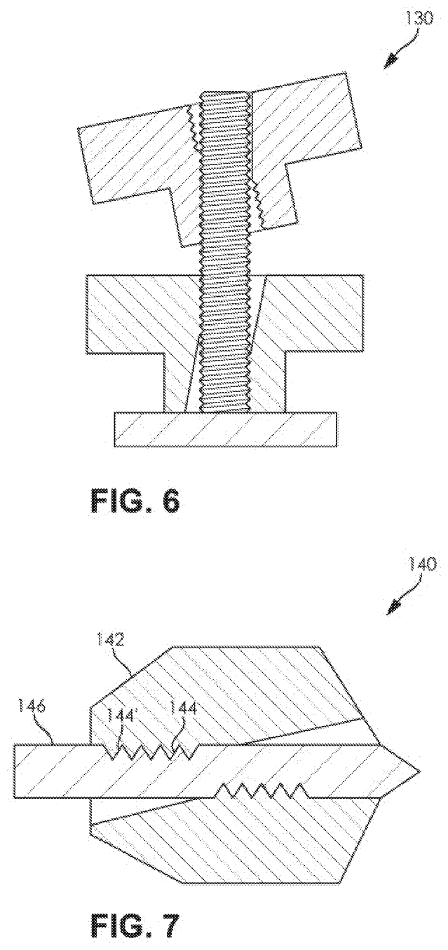

FIG. 7 is a side perspective cross-sectional view of a tunneler to central lead needle locking mechanism 140. The tunneler tip head 142 has a series of pawls 144 that are complementary to pawls 144′ along the side of the central needle 146. Under tunneler tip head 142 compression, the tip is locked to the serrated edges of the complementary pawls 144′ of the central needle 146. Pressure on the tunneler tip head 142 is relieved prior to advancing the central needle 146. Advancing the needle (and any hand motion) will permit the teeth of the pawls 144 to dislodge from the complementary pawls 144′ and permit advancement of the central needle 146.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention but is not meant to be a limitation upon the practice thereof. The following claims, including all equivalents thereof, are intended to define the scope of the invention.

1. A system for deploying a percutaneous conduit from within the skin of a patient in the subcutaneous layer comprising:
a tunneler having an elongated passer section, said elongated passer section having a proximal end terminated with an attachment feature for said percutaneous conduit and a distal end terminated in a tunneling head;
a central needle in said tunneling head;
a coaxial channel in said elongated passer section containing a wire for advancement of said central needle; and
a cylindrical trephine blade attached to the central needle, where said central needle is advanced through and pierces an outer layer of the patient's skin in an outward direction away from the patient, and said central needle defines a longitudinal axis of trephination or cylindrical cut of said cylindrical trephine blade in the patient's skin for placement of a medical appliance or a percutaneous access device (PAD).

2. The system of claim 1 further comprising a removable primary handle attached to said attachment feature for advancement of said elongated passer section through the subcutaneous layer to a desired exit site for placement of said medical appliance or said PAD.

3. The system of claim 1 further comprising a secondary handle for attachment to an engagement feature in said central needle, said secondary handle for pulling said elongated passer section out of the patient.

4. The system of claim 1 wherein said percutaneous conduit is treated with a primary coating.

5. The system of claim 4 wherein said primary coating comprises one or more of heparin, antibiotics, radiopaque agents, anti-thrombogenic agents, pro-thrombogenic agents, anti-proliferative agents, pro-proliferative agents, anti-angiogenic agents, and pro-angiogenic agents.

6. The system of claim 4 wherein said percutaneous conduit is treated with a secondary coating comprising one or more of polylactic acid, polyglycolic acid, polyethylene oxide, polycaprolactone, polydioxanones, combinations thereof, and co-polymers thereof.

7. The system of claim 1 wherein said percutaneous conduit is formed from a material that induces immunocompatible granulation tissue overgrowth thereon.

8. The system of claim 7 wherein the material is coated with one or more of poly-L-lysine (PLL), polylmethyl coguanidine-cellulose sulphate (PMCG)-CS/PLL-sodium alginate (SA), polyethylenimine, poly(dimethyldiallylammonium chloride), chitosan, polyacrylacid, carboxymethylcellulose, cellulose sulfate, pectin.

9. The system of claim 1 wherein the percutaneous conduit is one of a cardiac device driveline, a colostomy port, a vascular access conduit, a dialysis conduit, or any connection between an internal organ or cavity and an extracorporeal device.

10. The system of claim 1 further comprising a guidewire adapted to engage said needle.

11. The system of claim 1 further comprising a tracking system comprising a tracking computer and fiducial markers for determining the position and orientation of said needle relative to the skin of the patient.

12. A system for deploying a percutaneous conduit from within the skin of a patient in the subcutaneous layer comprising:
a tunneler having an elongated passer section, said elongated passer section having a proximal end terminated with an attachment feature for said percutaneous conduit and a distal end terminated in a tunneling head;
a central needle with a cam in a central channel of said tunneling head;
a lower chamber in the form of a semicircle off of said central channel that allows for partial upward movement of said cam to a first stop;
an upper chamber in the form of a semicircle off of said central channel that allows for partial upward movement of said cam to a second stop;
a coaxial channel in said elongated passer section containing a wire for advancement and twisting of said central needle; and
a cylindrical trephine blade actuated by said cam of said central needle in the upper chamber, where said central needle is advanced through and pierces an outer layer of the patient's skin in an outward direction away from the patient, and said central needle defines a longitudinal axis of trephination or cylindrical cut of said cylindrical trephine blade in the patient's skin for placement of a medical appliance or a percutaneous access device (PAD).

13. The system of claim 12 further comprising a removable primary handle attached to said attachment feature for advancement of said elongated passer section through the patient's subcutaneous layer to a desired exit site for placement of said medical appliance or said PAD.

14. The system of claim 12 further comprising a secondary handle for attachment to an engagement feature or eyelet in said central needle, said secondary handle for pulling said elongated passer section out of the patient.

15. The system of claim 12 wherein said percutaneous conduit is treated with a primary coating.

16. The system of claim 15 wherein said primary coating comprises one or more of heparin, antibiotics, radiopaque agents, anti-thrombogenic agents, pro-thrombogenic agents, anti-proliferative agents, pro-proliferative agents, anti-angiogenic agents, and pro-angiogenic agents.

17. The system of claim 16 wherein said percutaneous conduit is treated with a secondary coating comprising one or more of polylactic acid, polyglycolic acid, polyethylene oxide, polycaprolactone, polydioxanones, combinations thereof, and co-polymers thereof.

18. The system of claim 12 wherein said percutaneous conduit is formed from a material that induces immunocompatible granulation tissue overgrowth thereon.

19. (canceled)

20. The system of claim 1 wherein said tunneling head has a first set of pawls that are complementary to a second set of pawls along the side of the central needle; and
wherein when said tunneler head is under compression, said tunneler head is locked to the second set of pawls of the central needle.

21. A method of using the system of claim 1 for deploying said percutaneous conduit from within the skin of a patient in the subcutaneous layer comprising:
making a sub cut in the subcutaneous layer;
inserting the tunneler in the sub cut;
advancing the tunneling head to form a tunnel in the subcutaneous layer to an exit location;
deploying the central needle, or following a pre-positioned central guidewire, to pierce through the skin of the patient;
deploying the cylindrical trephine blade to make a cylindrical cut in the skin;
pulling or pushing the tunneler out of the patient to expose the driveline and positioning the medical appliance or PAD in the cylindrical cut; and
detaching the tunneler from the driveline.
